(19) United States
(12) Patent Application Publication
(10) Pub. No.: US 2009/0011029 A1
(43) Pub. Date: Jan. 8, 2009

(54) COLORLESS AND TRANSPARENT ANTIBIOTIC MATERIAL INCLUDING SILVER, AND A METHOD FOR THE PREPARATION OF IT

(75) Inventors: Hyung-Joon Lim, Incheon (KR); Young-Chul Yoo, Kyeongki-Do (KR); O-Sung Kwon, Seoul (KR); Doo-Yang Heo, Seoul (KR)

Correspondence Address: LEXYOUME IP GROUP, LLC, 5180 PARKSTONE DRIVE, SUITE 175, CHANTILLY, VA, 20151, US

(73) Assignee: SUKGYUNG A.T CO., LTD., Ansan (KR)

(21) Appl. No.: 11/577,002

(22) PCT Filed: Jun. 3, 2005
(86) PCT No.: PCT/KR05/01663

§ 371 Date: Apr. 10, 2007

(30) Foreign Application Priority Data
Oct. 12, 2004 (KR) 10-2004-0081479

(51) Int. Cl. A61K 9/14; A61K 33/38

(52) U.S. Cl. 424/489; 424/618

(57) ABSTRACT

Disclosed herein is a colorless and transparent antibiotic material including silver and a method of preparing the same. Specifically, the current invention pertains to a method of preparing a colorless and transparent antibiotic material including silver (Ag), which includes a) reacting a salt including a silver ion (Ag+) with a salt including a sulfate anion, to prepare a silver (Ag)-sulfate complex; and b) diluting the silver (Ag)-sulfate complex prepared in a) with water, and to an antibiotic material prepared using the method. Further, the current invention pertains to an antibiotic material including silver, which is harmless to the human body and exhibits disinfecting and antibiotic activities, and as well, is colorless and transparent and does not easily form colored oxides, unlike conventional silver-based antibiotic materials, and to a method of preparing such an antibiotic material. Thus, the colorless and transparent antibiotic material of this invention can be widely applied to manufacture industrial goods, such as nonwoven fabrics, packaging materials, etc., living goods, such as clothes, bedclothes, etc., and fiber goods.

### TECHNICAL FIELD

The present invention relates, in general, to a colorless and transparent antibiotic material including silver and a method of preparing the same. More particularly, the present invention relates to an antibiotic material composed mainly of silver, which is harmless to the human body, exhibits antibiotic and disinfecting effects, is colorless and transparent, and is stable to light, including ultraviolet (UV) light, thus not becoming colored or discolored upon the preparation of antibiotic goods using the same, and to a method of preparing such an antibiotic material.

### BACKGROUND ART

Generally, silver (Ag) is a metal component that exhibits antibiotic and disinfecting activities, and in particular, silver nanoparticles manifest superior antibiotic activity to pathogenic substances, such as bacteria or viruses, and causes no side effects in the human body. Further, silver nanoparticles have a small particle size, and thus prevents the generation of cracks upon the preparation of antibiotic goods. Hence, silver is widely used to manufacture fiber goods, such as clothes, bedclothes, shoe materials, etc., industrial goods, such as packaging materials, nonwoven fabrics, filters, adhesives, etc., and living goods, such as antibiotic sprays, functional cosmetics, etc.

Conventionally, an antibiotic material including silver, which is used for antibiotic goods, such as fibers or nonwoven fabrics, is based on a calcium phosphate support, a zirconium phosphate support, or a zeolite support, each of which has a metal ion, that is, a silver ion, substituted therein. Of these antibiotic materials, an antibiotic material based on the zeolite support has been widely developed.

In regard to the antibiotic material including silver, Korean Patent Application No. 1998-0012320 discloses a method of preparing antibiotic zeolite having high resin transparency and low water adsorption. In addition, Korean Patent Application No. 2002-0054807 discloses a method of preparing a silver nanoparticle/organic polymeric composite using radioactive rays and a silver nanoparticle/organic polymeric composite prepared using the method, and Korean Patent Application No. 2002-0055186 discloses a synthetic resin and silicone containing silver nanoparticles.

However, since a conventional antibiotic material including silver further comprises a surfactant, which functions as a protective colloid to inhibit the agglomeration of silver particles, and a reducing agent, which is necessary for the reduction of a metal salt, upon the preparation of the silver nanoparticles, the final antibiotic product including silver shows colors, for example, blue, yellow, brown, etc., thanks to the use of such additives. Further, when the silver nanoparticles are prepared using a microemulsion process or a polyol process, various additives should be used to easily disperse the silver particles, and thus, antibiotic goods have colors.

In addition, such a conventional antibiotic material including silver nanoparticles is blackened as represented by the following reaction when exposed to light, including UV light.

Ag+———Ag^{o}———Ag₂O (black)

Therefore, in order to solve the above problem, in the case where fibers or nonwoven fabrics are manufactured to have antibiotic activities, a colorless and transparent antibiotic material including silver, which does not change color and does not become discolored when exposed to light, such as UV light, is urgently required.

### DISCLOSURE

### Technical Problem

Accordingly, the present invention has been made keeping in mind the above problems occurring in the related art, and an object of the present invention is to provide a method of preparing an antibiotic material including silver (Ag), which is harmless to the human body, exhibits antibiotic and disinfecting activities, and is colorless and transparent, and an antibiotic material including silver thus prepared.

Another object of the present invention is to provide a method of preparing an antibiotic material including silver (Ag), which resists discoloration due to the reaction between a silver ion and light when exposed to light, including UV light, and an antibiotic material including silver thus prepared.

### Technical Solution

In order to achieve the above objects, the present invention provides a method of preparing a colorless and transparent antibiotic material including silver, comprising:

a) reacting a salt including a silver ion (Ag+) with a salt including a sulfate anion, to prepare a silver (Ag)-sulfate complex; and

b) diluting the silver (Ag)-sulfate complex prepared in a) with water.

In addition, the present invention provides a colorless and transparent antibiotic material including silver (Ag), prepared using the above method.

### ADVANTAGEOUS EFFECTS

The present invention provides a method of preparing an antibiotic material including silver (Ag), and an antibiotic material including silver thus prepared. According to the present invention, since the antibiotic material is composed mainly of silver, it is harmless to the human body and exhibits disinfecting and antibiotic activities. As well, unlike conventional silver-based antibiotic materials, the antibiotic material of the present invention is colorless and transparent, and thus, antibiotic goods manufactured using the antibiotic material of the present invention have no color problems.

In addition, even if the antibiotic material of the present invention is exposed to light, including UV light, it is stable and does not undergo deterioration, for example, discoloration resulting from easy formation of black oxide, such as Ag₂O, upon exposure to such light.

### BEST MODE

Hereinafter, a detailed description will be given of the present invention.

Leading to the present invention, intensive and thorough research into antibiotic materials having excellent antibiotic activities, being colorless and transparent, and being stable to light, including UV light, aiming to avoid the problems encountered in the related art, resulted in the finding that a salt including a silver ion (Ag+) may be reacted with a salt including a sulfate anion to prepare a predetermined complex, which can be confirmed to exhibit sufficient antibiotic activity, be colorless and transparent, and be stable to light, including UV light.

In the present invention, a method of preparing a colorless and transparent antibiotic material including silver is provided, which comprises a) reacting a salt including a silver ion (Ag+) with a salt including a sulfate anion, to prepare a silver (Ag)-sulfate complex; and b) diluting the silver (Ag)-sulfate complex prepared in a) with water.

The salt including a silver ion (Ag+) used in a) is not particularly limited in the present invention, as long as it may provide a silver ion (Ag+) in a reaction solvent. In particular, such a salt is preferably silver nitrate (AgNO₃) or silver acetate (AgCH₃COO).

Likewise, the salt including a sulfate anion is not particularly limited in the present invention, as long as it may provide a sulfate anion in a reaction solvent. Such a salt is preferably selected from the group consisting of sodium sulfate, sodium thiosulfate, sodium pyrosulfate, sodium sulfite, sodium pyrosulfite, potassium sulfate, potassium thiosulfate, potassium pyrosulfite, potassium sulfite, potassium pyrosulfite, ammonium sulfate, ammonium thiosulfate, ammonium sulfite, and mixtures thereof.

In addition, the silver (Ag)-sulfate complex obtained in a) may be prepared by reacting the salt including a silver ion (Ag+) with the salt including a sulfate anion in a reaction solvent. As such, the silver (Ag)-sulfate complex is preferably silver thiosulfate.

Further, the reaction solvent is water or an organic solvent, and is preferably water.

Specifically, the preparation of the silver (Ag)-sulfate complex in a) is preferably conducted by mixing the aqueous solution of the salt including a silver ion (Ag+) with the aqueous solution of the salt including a sulfate anion, and then aging the mixture at 60˜80° C. for a time ranging from 30 min to 2 hr. In this way, when the aging process is carried out in the above range, the resultant complex is highly stable to light, including UV light, and to ultrapure water used for dilution in b). Preferably, the aqueous solution of the salt including a silver ion (Ag+) has a concentration of 0.1˜1.0 mol/L, and the aqueous solution of the salt including a sulfate anion has a concentration of 1.0˜5 mol/L. The silver (Ag)-sulfate complex may have an average particle size of 10 nm or less. More preferably, the aqueous solution of the salt including a silver ion (Ag+) has a concentration of 0.3˜0.5 mol/L, and the aqueous solution of the salt including a sulfate anion has a concentration of 3˜5 mol/L.

Subsequently, the silver (Ag)-sulfate complex prepared in a) is diluted with water to have a desired concentration. Thereby, a colorless and transparent antibiotic material including silver may be obtained at a desired concentration. At this time, ultrapure water, which is passed through an ion exchange resin and then undergoes tertiary distillation using a distillation apparatus, is preferably used to decrease the influence of impurities.

The antibiotic material including silver has excellent antibiotic activity and light stability, when the content of the silver (Ag)-sulfate complex formed in a) is 1˜10,000 ppm. If the above content is less than 1 ppm, antibiotic and disinfecting activities become insignificant. On the other hand, if the content exceeds 10,000 ppm, a blackening phenomenon may occur in a short time upon exposure to light, including UV light.

In the method of the present invention, when the silver (Ag)-sulfate complex is prepared in a), a salt including metal having antibiotic activity may be additionally used along with the salt including a silver ion (Ag+).

The salt including metal having antibiotic activity, which may be additionally used, is not particularly limited in the present invention, as long as it is able to provide a cation salt of copper, nickel, platinum, palladium, or ruthenium, each of which has antibiotic or deodorizing activities. Preferably, the above salt is exemplified by a water-soluble metal salt, including copper nitrate (Cu(NO₃)₂), copper acetate (Cu(CH₃COO)₂), copper chloride (CuCl₂), nickel nitrate (Ni(NO₃)₂), nickel acetate (Ni(CH₃COO)₂), nickel sulfate (NiSO₄), chloroplatinic acid (H₂PtCl₆), hydrogen tetrachloroaurate (HAuCl₄) or palladium chloride (PdCl₂); an organic solvent-soluble metal salt, including acetylacetonates, such as nickel acetylacetonate or copper acetylacetonate; or a hydrolyzable metal salt, including alkoxides, such as nickel ethoxide or copper ethoxide, depending on the type of the reaction solvent. Preferably, the maximum amount of the additionally used metal salt is 20 mol %, based on the total amount of cations including a silver ion and a metal ion, in consideration of disinfecting power and average particle size.

In addition, the present invention provides a colorless and transparent antibiotic material including silver, prepared using the above method. The antibiotic material including silver contains 10˜10,000 ppm silver (Ag)-sulfate complex, and preferably, 100˜1,000 ppm silver (Ag)-sulfate complex.

Further, in the antibiotic material including silver of the present invention, the silver (Ag)-sulfate complex preferably has an average particle size not exceeding 10 nm. If the average particle size exceeds 10 nm, light stability is worsened, and thus, the antibiotic material may become discolored. Thus, the average particle size is preferably 2˜5 nm, and more preferably 3.5˜4.5 nm, with a standard deviation of 5 Å or less.

### MODE FOR INVENTION

Hereinafter, the present invention is specifically explained using the following examples, which are set forth to illustrate, but are not to be construed to limit the present invention.

### EXAMPLE 1

A 0.4 mol/L aqueous silver nitrate (AgNO₃) solution was added with a 1.5 mol/L aqueous sodium sulfite solution and then with 3 mol/L sodium thiosulfate, and thereafter was allowed to react at 60˜80° C. for 1 hr, to prepare a silver-sulfate complex.

The silver-sulfate complex was diluted with ultrapure water, which had been passed through an ion exchange resin and then undergone tertiary distillation using a distillation apparatus, so that the solid content of the silver-sulfate complex was 1 wt %, to prepare a final colorless and transparent antibiotic solution including silver.

According to a particle size analysis, the antibiotic solution including silver thus prepared had an average particle size of 3.9 nm, and a standard deviation of 4 Å.

The antibiotic solution thus prepared was assayed for bacterial reduction according to a pressurization close adhesion method. The bacterial reduction was measured in such a way that test strains (Escherichia coli ATCC 25922, Staphylococcus aureus ATCC 6538, and Salmonella typhmurium KCTC 1925) were static cultured on the antibiotic solution including silver having a surface area of 60 cm² at 25° C. for 18 hr, followed by counting the number of cells. The results are given in Table 1 below.

TABLE 1

| | Escherichia | | | | | |
|---|---|---|---|---|---|---|
| | coli ATCC | | Staphylococcus | | Salmonella | |
| | 25922 | | aureus ATCC 6538 | | typhmurium KCTC 1925 | |
| | | Coating in | | Coating | | Coating in |
| | Blank | Ex. 1 | Blank | in Ex. 1 | Blank | Ex. 1 |
| Immediately | 1.5 × 10° | 1.5 × 10° | 1.4 × 10° | 1.4 × 10° | 1.2 × 10° | 1.2 × 10° |
| After | | | | | | |
| Contact | | | | | | |
| After | 6.9 × 10° | <10 | 6.7 × 10° | <10 | 5.9 × 10° | <10 |
| Culture | | | | | | |
| for 18 hr | | | | | | |
| Bacterial | — | 99.9 | — | 99.9 | — | 99.9 |
| Reduction | | | | | | |
| (%) | | | | | | |

* Blank: No antibiotic solution including silver.

As is apparent from Table 1, the antibiotic solution including silver of Example 1 was quite different in bacterial reduction from that of the blank. From this result, the antibiotic solution including silver of Example 1 was confirmed to exhibit excellent antibiotic activity.

### EXAMPLE 2

The present example was conducted under the same test conditions as in Example 1, with the exception that an antibiotic solution, in which a solid content of silver-sulfate complex was 0.01 wt % (100 ppm), was prepared. The results are given in Table 2 below.

TABLE 2

| | Escherichia | | Staphylococcus | | Salmonella | |
|---|---|---|---|---|---|---|
| | coli | | aureus | | typhmurium | |
| | ATCC 25922 | | ATCC 6538 | | KCTC 1925 | |
| | | Coating | | Coating in | | Coating |
| | Blank | in Ex. 2 | Blank | Ex. 2 | Blank | in Ex. 2 |
| Immediately | 1.4 × 10° | 1.4 × 10° | 1.5 × 10° | 1.5 × 10° | 1.6 × 10° | 1.6 × 10° |
| After | | | | | | |
| Contact | | | | | | |
| After | 5.9 × 10° | <10 | 6.5 × 10° | <10 | 7.0 × 10° | <10 |
| Culture | | | | | | |
| for 18 hr | | | | | | |
| Bacterial | — | 99.9 | — | 99.9 | — | 99.9 |
| Reduction | | | | | | |
| (%) | | | | | | |

* Blank: No antibiotic solution including silver.

As is apparent from Table 2, the antibiotic solution including silver of Example 2 was quite different in bacterial reduction from that of the blank. From this result, the antibiotic solution including silver of Example 2 was confirmed to exhibit excellent antibiotic activity.

In addition, the antibiotic solution of Example 1, in which the solid content of the silver-sulfate complex was 1 wt % (10,000 ppm), and the antibiotic solution of Example 2, in which the solid content of the silver-sulfate complex was 0.01 wt % (100 ppm), were subjected to a discoloration test when exposed to solar light and also when placed in an indoor room over time. The results are given in Table 3 below.

TABLE 3

| | | Immediately | After | | | |
|---|---|---|---|---|---|---|
| | | After | 10 | After 20 | After | After 50 |
| | | Synthesis | Days | Days | 30 Days | Days |
| Blank | Ex. 1 | No Change | — | — | — | — |
| (Darkroom) | Ex. 2 | No Change | — | — | — | — |
| Indoor | Ex. 1 | No Change | — | — | — | — |
| Means | Ex. 2 | No Change | — | — | — | — |
| (Fluorescent | | | | | | |
| Lamp) | | | | | | |
| Outdoor | Ex. 1 | No Change | — | Light | Dark | Complete |
| Means | | | | Black | Black | Black |
| (Solar | Ex. 2 | No Change | — | — | — | — |
| Light) | | | | | | |

* Blank: Solution was loaded into a brown bottle and stored in a dark cold space.

As is apparent from Table 3, the antibiotic solutions of Examples 1 and 2 did not discolor for up to 30 days in the room. After 50 days, it was confirmed that only the solution of Example 1 (solid content: 10,000 ppm) became discolored to light black, and the solution of Example 2 (solid content: 100 ppm) was maintained colorless and transparent without a change of color.

Further, when the antibiotic solutions of Examples 1 and were exposed to solar light, they did not discolor for up to 10 days. After 20 days, the solution of Example 1 became gradually discolored and was completely blackened after 50 days. However, the solution of Example 1 was considered to be remarkably stable to light, compared to conventional antibiotic solutions including silver, which discolored immediately after exposure to solar light. Moreover, it was noted that the color of the solution of Example 2 did not change even if the exposure time of the above solution to solar light exceeded 50 days.

### INDUSTRIAL APPLICABILITY

As described hereinbefore, the present invention provides a method of preparing an antibiotic material including silver (Ag), and an antibiotic material including silver (Ag) prepared using the method. According to the present invention, since the above antibiotic material is composed mainly of silver, it is harmless to the human body and exhibits disinfecting and antibiotic activities. As well, unlike conventional silver-based antibiotic materials, the antibiotic material of the present invention is colorless and transparent, and thus, antibiotic goods manufactured using the antibiotic material of the present invention have no color problems.

In addition, even if the antibiotic material of the present invention is exposed to light, including UV light, it is stable and does not cause side effects, for example, discoloration resulting from easy formation of black oxide, such as Ag₂O, upon exposure to such light.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of preparing a colorless and transparent antibiotic material including silver, comprising:
a) reacting a salt including a silver ion (Ag+) with a salt including a sulfate anion, to prepare a silver (Ag)-sulfate complex; and
b) diluting the silver (Ag)-sulfate complex prepared in a) with water.

2. The method according to claim 1, wherein the preparation of the silver (Ag)-sulfate complex in a) is conducted by mixing an aqueous solution of the salt including a silver ion (Ag+) with an aqueous solution of the salt including a sulfate anion, to obtain a mixture, which is then aged at 60˜80° C. for a time ranging from 30 min to 2 hr.

3. The method according to claim 2, wherein the aqueous solution of the salt including a silver ion (Ag+) has a concentration of 0.1˜1.0 mol/L, and the aqueous solution of the salt including a sulfate anion has a concentration of 1.0˜5 mol/L.

4. The method according to claim 1, wherein the dilution of the silver (Ag)-sulfate complex in b) is conducted by using ultrapure water, so that content of the silver (Ag)-sulfate complex is 10˜10,000 ppm.

5. The method according to claim 1, wherein the salt including a silver ion (Ag+) is silver nitrate (AgNO₃) or silver acetate (AgCH₃COO).

6. The method according to claim 1, wherein the salt including a sulfate anion is selected from the group consisting of sodium sulfate, sodium thiosulfate, sodium pyrosulfate, sodium sulfite, sodium pyrosulfite, potassium sulfate, potassium thiosulfate, potassium pyrosulfite, potassium sulfite, potassium pyrosulfite, ammonium sulfate, ammonium thiosulfate, ammonium sulfite, and mixtures thereof.

7. The method according to claim 1, wherein a salt including metal having antibiotic activity is further used along with the salt including a silver ion (Ag+) in a).

8. The method according to claim 7, wherein the salt including metal having antibiotic activity is selected from the group consisting of copper nitrate (Cu(NO₃)₂), copper acetate (Cu (CH₃COO)₂), copper chloride (CuCl₂), nickel nitrate (Ni(NO₃) 2), nickel acetate (Ni(CH₃COO) 2), nickel sulfate (NiSO₄), chloroplatinic acid (H₂PtCl₆), hydrogen tetrachloroaurate (HAuCl₄), palladium chloride (PdCl₂), nickel acetylacetonate, copper acetylacetonate, nickel ethoxide, copper ethoxide, and mixtures thereof.

9. A colorless and transparent antibiotic material including silver, prepared using the method of claim 1.

10. The antibiotic material according to claim 9, wherein a silver (Ag)-sulfate complex is included in a content of 10˜10,000 ppm.

11. The antibiotic material according to claim 9, wherein the silver (Ag)-sulfate complex has an average particle size of 2˜5 nm.

12. The antibiotic material according to claim 9, wherein the silver (Ag)-sulfate complex is silver thiosulfate.
